(19) United States
(12) Patent Application Publication
(10) Pub. No.: US 2005/0169929 A1
(43) Pub. Date: Aug. 4, 2005

(54) Use of a vaccine for active immunization against cancer

(76) Inventors: Gottfried Himmler, Vienna (AT); Hans Loibner, Vienna (AT); Hellmut Samonigg, Graz (AT)

Correspondence Address: BIRCH STEWART KOLASCH & BIRCH, PO BOX 747, FALLS CHURCH, VA, 22040-0747, US

(21) Appl. No.: 10/515,146

(22) PCT Filed: May 20, 2003
(86) PCT No.: PCT/AT03/00148

(30) Foreign Application Priority Data
May 21, 2002 (AT) A 774/2002

(51) Int. Cl. A61K 39/395; A61K 39/00

(52) U.S. Cl. 424/155.1; 424/277.1

(57) ABSTRACT

Disclosed is the use of a vaccine based on a tumor-associated antigen, its epitope, mimotope, specific or anti-idiotypic antibody, for preparing a medicament for the prophylactic and/or therapeutic active immunization against cancer, in combination with chemotherapy.

The invention relates to the use of a vaccine based on a tumor-associated antigen, its epitope, mimotope, specific or anti-idiotypic antibody, for preparing a medicament, as well as to a kit for the prophylactic and/or therapeutic active immunization against cancer.

Tumor-associated antigens (TAA) often are the basis for the development of immunotherapeutic agents for the prophylaxis and/or treatment of cancer. TAA are structures which preferably are expressed on the cell membrane of tumor cells, thereby enable a differentiation relative to non-malignant tissue and thus are viewed as targets for the diagnostic and therapeutic applications of specific antibodies. Examples of tumor-associated carbohydrate structures are the Lewis antigens which are highly expressed in many epithelial types of cancer. Among them are Lewis x-, Lewis b- and Lewis y-structures, as well as sialylated Lewis x-structures. Other carbohydrate antigens are GloboH structures, KH1, Tn antigen, TF antigen, the alpha-1,3-galactosyl epitope (Elektrophoresis (1999), 20: 362; Curr. Pharmaceutical Design (2000), 6: 485, Neoplasma (1996), 43: 285).

Further TAAs are proteins which are particularly highly expressed on cancer cells, such as, e.g., CEA, TAG-72, MUC1, Folate Binding Protein A-33, CA125, Ep-CAM and PSA.

Direct therapeutic applications of antibodies against TAA are based on passive immunotherapies, i.e., a specific antibody is systemically administered to cancer patients in a suitable amount and has only a therapeutic action as long as its concentration in the organism is high enough for this. The biological half-life of such agents depends on their structures and ranges from a few hours to several days. Therefore, it is necessary to provide repeated applications. When using xenogenic antibodies (e.g. murine monoclonal antibodies, MAB), this, however, will lead to undesired immune reactions which can neutralize a possible therapeutic activity and may cause dangerous side effects (anaphylactic reactions). Therefore, such immune therapeutic agents can be administered for a limited time only.

A different approach for the immunotherapy of cancer is based on the selective activation of the immune system of cancer patients to fight malignant cells. This is attempted by the most varying forms of cancer vaccines. Among them are vaccinations with autologous or allogenic tumor cells, chemically or molecular-biologically modified autologous or allogenic tumor cells, isolated TAAs or TAAs prepared with the help of chemical or molecular-biological methods, peptides derived therefrom, more recently also vaccinations with DNA which codes for TAA or structures derived therefrom, etc. An alternative method is based on the use of anti-idiotypic antibodies for vaccinating against cancer. Suitable anti-idiotypic antibodies may mimic a TAA immunologically. As foreign proteins (e.g. murine antibodies, goat antibodies etc.) they induce a strong immune response in humans after a vaccination-in contrast to the human tumor antigens proper which, being self-structures, often are only little immunogenic. Therefore, anti-idiotypic antibodies can be used as an immunogenic substitute of a tumor antigen for vaccination purposes.

In contrast to the passive immunotherapies using anti-tumor antibodies, in principle very small amounts of a suitable vaccine will suffice for the active specific cancer immunotherapy so as to induce an immunity for months or even years which, when it becomes weaker, can be boosted by booster vaccinations. Moreover, in an active immunization both a humoral and also a cellular immunity can be induced whose interaction may yield an effective protective action.

Summing up, so far the use of antibodies or their derivatives in the cancer immunotherapy has essentially been based on two principles:
Passive therapy with antibodies or their derivatives which are directed against TAA. In this instance, tumor cells are relatively specifically destroyed (Immunology Today (2000), 21: 403-410; Curr. Opin. Immunol. (1997), 9: 717).
Active immunization (vaccination) with TAA, or antibodies, respectively, or their derivatives, which are directed against the idiotype of antibodies having a specificity against TAA. The active vaccination triggers an immune response against TAA. This immune response thus is also directed against the corresponding tumor cells (Ann. Med. (1999), 31: 66; Immunobiol. (1999), 201: 1).

In the course of the discovery and the subsequent characterization of the most varying TAA it has been found that they have important functions for cancer cells. They allow the degenerated cells to have characteristic properties of the malignant phenotype, such as, e.g., an increased adhesion ability, which is of great importance for establishing metastases. However, such antigens may very well be expressed on normal cells in certain stages, where they are responsible for the normal functions of these cells. Without a claim to completeness, a few examples of such antigens should be mentioned here:
N-CAM (neuronal Cell Adhesion Molecule) which is often expressed on tumors of neuronal origin and causes homophilic adhesion (J. Cell Biol. 118 (1992), 937).
The Lewis Y carbohydrate antigen which appears on most of the tumors of epithelial origin, but also plays an important role in the fetal development of epithelial tissues. It has been shown that the expression of this antigen in lung cancer is highly associated with an unfavorable prognosis, since Lewis Y-positive cancer cells apparently have a higher metastatic potential (N. Engl. J. Med. 327 (1992), 14).
CEA (Carcino Embryonic Antigen) which often appears on epithelial tumors of the gastro-intestinal tract and has been identified as a self-adhesion molecule (Cell 57 (1989), 327).
Ep-CAM (Epithelial Cell Adhesion Molecule) which is expressed on almost all tumors of epithelial origin, but also appears on many normal epithelia, which has been characterized as a self-adhesion molecule and therefore can be classified as a pan-epithelial adhesion antigen (J. Cell Biol. 125 (1994), 437).

In WO 00/41722-A1, the use of antibodies directed against human cellular membrane proteins for the active immunization of cancer patients has been described. Among them is a vaccine containing the murine monoclonal antibody HE2, directed against the Ep-CAM molecule.

Anti-Ep-CAM antibodies are also used for passive immunotherapy. EP 0 755 683-A1 describes the treatment with a high antibody dose in combination with chemotherapy, radiotherapy or surgery.

Likewise, an antibody directed against the “Epidermal Growth Factor” receptor-2 (HER-2) for passive immunotherapy is used in combination with chemotherapy (Anticancer Drugs 2001, 12 Suppl 4: pp. 3-10).

Passive immunotherapy has also been described in WO 01/07082, also in combination with a chemotherapeutic agent which is to lead to the arrest of Ep-CAM expressing cells in the S or G2/M cell cycle phase.

In Current Opinion in Molecular Therapeutics 2000, 2(4): 453-458 and U.S. Pat. No. 5,747,048, however, the active immunization with a vaccine has been described which contains a synthetic mimic of the mucin-associated glycan epitope SialylTn conjugated to a carrier molecule. This vaccine actively induces an immune response, primarily if the patient has previously been subjected to a chemotherapy.

In U.S. Pat. No. 5,792,455 and in Clin. Cancer Res., 1999, 5, 1319-1323, the use of a monoclonal IgG2b antibody, BEC2, which constitutes a mimic for GD3, is described for the treatment of melanomas, or small-cell lung carcinoma (SCLC), i.a. in combination with chemotherapy. The BEC2 antibody is administered with BCG/Bacillus Calmette-Guerin, which is a very highly immunogenic adjuvant. By the treatment with the antibody in combination with adjuvants which have a highly immunogenic effect, here partly very grave side reactions, such as fever and a rise of the glucose level, are caused.

Chemotherapy itself is used to treat cancer patients so as to combat the dividing tumor cells by chemical means. Disseminated tumor cells which are in their dormant stage, however, are not attacked by the chemotherapy (“minimal residual disease”), yet they are associated with the formation of metastases. Therefore, professional circles are increasingly dealing with immunotherapy for combatting disseminated tumor cells.

The present invention now has as its object to improve the use of vaccines for an active immunization of cancer patients.

This object is achieved by providing the embodiments set forth in the claims.

According to the invention, a vaccine based on a tumor-associated antigen (TAA), its epitope, mimotope, specific or anti-idiotypic antibody is used for preparing a medicament which is employed for the prophylactic and/or therapeutic active immunization against cancer in combination with chemotherapy.

The TAA preferably is selected from the group of the peptides or proteins, in particular Ep-CAM, N-CAM, CEA and T-cell peptides, the carbohydrates, in particular Lewis Y, sialyl-Tn, GloboH, and the glycolipids, in particular GD2 and GM2, and epitopes or mimotopes of these antigens. The TAAs used in the vaccine according to the invention preferably induce a functional immune response directed against tumor cells. In doing so, not only tumor cells during the cell division, but also in the dormant state, are to be attacked for an effective treatment of the “minimal residual disease”, or the reduction of the metastasizing potential.

In any event, the inventive combined immunotherapy aims at functionally supporting the immune system so as to bind the induced antibodies not only to the tumor cells, but also to reduce them by a humoral or cellular immune response. The competitive binding of an antibody to a receptor of a tumor cell having tyrosine kinase activity is considered to be insufficient. Finally, within the scope of an immunotherapy, not only a receptor is to be blocked and the enzymatic cell division is to be stopped, but also tumor cells are to be eliminated, such as by lysis.

Within the scope of the present invention, therefore, those structures of a possible TAA are excluded which merely represent receptor proteins with tyrosine kinase activity whose immunocomplex formation is to inhibit the enzymatic reaction for cell division. Receptor proteins are derived, e.g., from HER-2 or from the similar receptor c-erbB-2, which have a tyrosine kinase activity and support cell division. Such receptor protein-TAAs, or their mimic or anti-idiotypic antibodies, respectively, therefore attack the dividing cell, just like chemotherapy does. As an example thereof, gp75 protein derived from c-erbB-2 receptor is described in EP 1006194 which, either alone or in combination with a chemotherapy, is to stop cell division.

The epitopes imitate or primarily comprise domains of a natural, homologous or derivatized TAA. They are comparable with the TAAs at least by their primary structure and, possibly, by their secondary structure. The epitopes may, however, also differ completely from TAAs in this respect and may imitate components of a TAA, primarily of proteinaceous or carbohydrate antigens, purely by the similarity of spatial (tertiary) structures. The tertiary structure of a molecule alone may thus form a mimic which will trigger the immune response to a certain TAA.

As a rule, it will have to be assumed that by an antigen which imitates a proteinaceous epitope of a tumor-associated antigen, a polypeptide of at least five amino acids is to be understood.

Preferred TAAs are derived from antigens which are specific of epithelial tumors and increasedly occur e.g. in breast cancer, cancer of the stomach and intestines, of the prostate, pancreas, ovaries and the lungs. Among the preferred TAAs are those which primarily trigger a humoral immune response, i.e. a specific antibody formation, in vivo. On the other hand, also those antigens can be chosen according to the invention which generate a T-cell specific immune response. Among them are primarily intracellular structures, or T-cell peptides, respectively.

According to the invention, preferably epithelial cancers, such as breast cancer, cancer of the stomach and intestines, of the prostate, pancreas, ovaries and the lung, e.g. SCLC (“small cell lung cancer”) and NSCLC (“non small cell lung cancer”) are treated.

Further preferred proteinaceous TAAs which are particularly expressed on the cancer cells of solid tumors are, e.g., TAG-72, MUC1, Folate Binding Protein A-33, CA125, PSA, MART etc. (cf. e.g., Sem. Cancer Biol. 6 (1995), 321). Furthermore, also so-called T-cell epitope peptides (Cancer Metastasis Rev. 18 (1999), 143; Curr. Opin. Biotechnol. 8 (1997), 442; Curr. Opin. Immunol. 8 (1996), 651) or mimotopes of such T-cell epitopes (Curr. Opin. Immunol. 11 (1999), 219; Nat. Biotechnol. 16 (1998), 276-280) may serve. Suitable TAAs are expressed at least in 20%, preferably at least in 30% of the cases of tumor cells of a certain type of cancer, more preferred in at least 40%, in particular in at least 50% of the patients.

Carbohydrate epitopes preferred according to the invention are tumor-associated carbohydrate structures, such as the Lewis antigens, e.g. Lewis x-, Lewis b- and Lewis y-structures, as well as sialylated Lewis x-structures. Furthermore, also GloboH structures, KH1, Tn antigen or sialyl-Tn, Tf-antigen, the alpha-1,3-galactosyl epitope are preferred carbohydrate antigen structures within the scope of the present invention.

In a particular embodiment, according to the invention at least two equal or different epitopes of an adhesion protein, e.g. a homophilic cellular membrane protein, such as Ep-CAM, are provided, or imitated, respectively, in the vaccine. Thus, by the active immunization, a plurality of antibodies with a specificity for the same molecule, yet for different Ep-CAM binding sites, can be generated.

The vaccine can also comprise a glycosilated antibody, particularly if the glycosilation itself can also imitate an epitope of a carbohydrate epitope of a TAA. It has been found that an epitope of an Ep-CAM protein and an epitope of a Lewis carbohydrate component, e.g. of Lewis Y, can be combined preferably with the SialylTn carbohydrate antigen. In particular, a Lewis Y glycosilated antibody having a specificity for an Ep-CAM structure constitutes a very good immunogen in a vaccine formulation used according to the invention. This antibody may particularly well imitate cellular tumor antigens, and accordingly, it causes the desired immune response for the inhibition of epithelial tumor cells.

According to the invention, the vaccine may be used for an active immunization, and therefore it is administered in small amounts only. Thus, no particular side effects are expected, particularly no fever and no increase in the glucose level, even if the immunogenic active substance used according to the invention is derived from a non-human species, such as a murine antibody. However, it is assumed that a recombinant, chimeric as well as a humanized or human active substance combined with murine and human components will be particularly suitable for an administration to humans. On the other hand, a murine portion in the active substance by being foreign may additionally provoke the immune response in humans.

By the term “antibody”, antibodies of all types are to be understood, in particular polyclonal or monoclonal antibodies or also chemically, biochemically or molecular-biologically prepared antibodies.

Although, naturally, the vaccine used according to the invention may contain an active substance derived from a native antibody, which possibly has been isolated from an organism or patient, often an antibody-derivative is used which preferably is selected from the group of antibody fragments, conjugates or homologues, but also complexes and adsorbates. In any case, it is preferred for the antibody derivative to contain at least parts of the Fab fragment, preferably together with at least parts of the F(ab′)2-fragment, and/or parts of the hinge-region and/or of the Fc-part of a lambda or kappa antibody. Furthermore, also a single-chain antibody derivative, such as a so-called single chain antibody may be used in a vaccine as defined by the invention. Preferably, an antibody of the type of an immunoglobulin, e.g. an IgG, IgM or IgA, is used. Particularly preferably an IgG2a-antibody is used, since IgG2a antibodies exhibit a particularly good complement activation, resulting in an increased immunogenicity of the vaccine. This, moreover, has the advantage that the content of antibody in the vaccine can be further reduced.

According to the invention, preferably also a vaccine is used which comprises an antibody or an antibody derivative directed against a tumor-associated antigen, i.e. an ab1. The specificity of the antibody preferably is chosen from among the above-mentioned groups of the TAAs, in particular selected from the group of Ep-CAM, N-CAM, CEA and Lewis Y or sialyl-Tn antigens. A particularly preferred antibody is the HE2-antibody, as described in WO 00/41722. This antibody is directed against the Ep-CAM protein; however, as an immunogen in a vaccine formulation, it may also trigger an anti-Ep-CAM immune response.

A particularly good immunogen for Ep-CAM is, e.g., an anti-Ep-CAM antibody that imitates or comprises at least one or at least two Ep-CAM epitopes, e.g. by its Ep-CAM-like idiotype. Such an antibody is, e.g., derived from an anti-Ep-CAM antibody from WO 00/41722.

A particularly preferably used vaccine contains an antibody which specifically binds an antibody. The tumor vaccine thus contains in particular anti-idiotypic antibodies, i.e. ab2, for an active immunization. Antiidotypic antibodies used according to the invention preferably again recognize the idiotype of an antibody which is directed against a TAA. In this manner, already an epitope of a TAA on the paratope of the anti-idiotypic antibody is formed as a mimic for the TAA.

Here, too, the preferred selection is made from the above-indicated groups of the TAAs. For example, an anti-idiotypic antibody against glycan-specific antibodies is used, such as an anti-idiotypic antibody which recognizes the idiotype of an anti-Lewis Y antibody, as described in EP 0 644 947, e.g.

The vaccine used according to the invention advantageously is provided in a suitable formulation. Preferred are such formulations with a pharmaceutically acceptable carrier. This comprises, e.g., auxiliary substances, buffers, salts, preservatives. The vaccine may, e.g., be used for the prophylaxis and therapy of cancer-associated conditions, such as metastasis formation and minimal residual disease of cancer patients. In this instance, antigen-presenting cells are specifically modulated in vivo or also ex vivo so as to specifically generate the immune response against the TAAs and the tumor cells.

A vaccine formulation preferably used according to the invention contains the immunogenic active substance in most cases only at low concentrations, such as in an immunogenic amount ranging from 0.01 μg to 10 mg. Depending on the nature of the TAA, its epitope, mimotope, specific or anti-idiotypic antibody, depending on the use of species-foreign sequences or derivatization, but also depending on the auxiliary agents or adjuvants, respectively, used, the suitable immunogenic dose will be chosen, e.g. in the range of from 0.01 μg to 750 μg, preferably 100 μg to 500 μg. A depot vaccine which is to be delivered to the organism over an extended period of time may, however, also contain much higher antibody amounts, such as from at least 1 mg to up to more than 10 mg. The concentration will depend on the administered amount of the liquid or suspended vaccine. A vaccine usually is provided in ready-to-use syringes having a volume of from 0.01 to 1 ml, preferably 0.1 to 0.75 ml, of the concentrated solution, or suspension, respectively.

The vaccine used according to the invention preferably presents the immunogen in a pharmaceutically acceptable carrier which is suitable for a subcutaneous, intramuscular or also intradermal or transdermal administration. A further type of administering is via the mucosal pathway, such as a vaccination by nasal or peroral administration. If solids are employed as auxiliary agents for the vaccine formulation, e.g. an adsorbate or a suspended mixture of the antibody with the auxiliary agent is administered. In special embodiments, the vaccine is administered as a solution, or liquid vaccine, respectively, in an aqueous solvent.

Preferably, one or more vaccine units of the tumor vaccine are already provided in suitable ready-to-use syringes. As an antibody is relatively stable as compared to the TAAs, vaccines based on antibodies or their derivatives have the essential advantage that they can be put on the market as a storage-stable solution or suspension in a ready-to-use form. However, a content of preserving agents, such as thimerosal or other preserving agents with an improved tolerance is not necessary, may, however, be provided in the formulation for a longer durability at storage temperatures of from refrigerator temperatures up to room temperature. The vaccine used according to the invention may, however, also be provided in frozen or lyophilized form which may be thawed, or reconstituted, respectively, when needed.

In any case, it has proven suitable to increase the immunogenicity of the active substance used according to the invention by using adjuvants in the vaccine formulation. Vaccine adjuvants suitable therefor preferably are aluminum hydroxide (Alu gel) or -phosphate, also growth factors, lymphokines, cytokines, such as IL-2, IL-12, GM-CSF, gamma interferon or complement factors, such as C3d, furthermore liposome preparations, and also formulations with additional antigens, against which the immune system has already produced a strong immune response, such as tetanus toxoid, bacterial toxins, such as Pseudomonas exotoxins and derivatives of Lipid A.

An adjuvant which allows for the medicament to be administered without any side effects is preferred. The term side effects encompasses, e.g., an increased glucose level or fever; local reddenings at the site of administration or slight swellings are not considered to be special side effects.

To formulate the vaccine, also further known methods for conjugating or denaturing vaccine components may be used so as to further increase the immunogenicity of the active substance.

Particular embodiments of the vaccine used according to the invention comprise further vaccination antigens, in particular additional anti-idiotypic antibodies, also mixtures of immunogenic antibodies with various antibodies which are simultaneously administered.

The use according to the invention of the vaccine is preferably at the beginning of the chemotherapy. Vaccination may be started already at the time of a possible surgery or even before the surgical removal of tumor tissue. Thus, protection against a possible dissemination of tumor cells can be built up by specific antibodies already at the time of the surgery. Preferably, chemotherapy is started within 1 to 2 weeks. A vaccination simultaneously with and/or during the chemotherapy is also preferred for practical reasons. The patient is already under clinical treatment, the additional therapeutic measures are easy to carry out.

If the immunotherapy is already effected on the first day of the chemotherapy or within the first 2 to 3 days, the immune system can be activated already at an early point of time, even before the organism has been adversely affected by the chemotherapy. Chemotherapy does, in fact, have side effects, such as that of a weakened immune system, which makes the patient increasingly infection-prone. Precisely for this reason it has been surprising that the immunotherapy can successfully be used immediately before and during the chemotherapy. Thus, it could be observed that the immune response after the inoculation with a tumor vaccine could be induced on the first day, several hours prior to the beginning of the chemotherapy, to the same extent as without a chemotherapeutical treatment. In any event, the serum content of immunoglobulins and vaccination antigen-specific antibodies was persistently increased, irrespective of a chemotherapy. There were even signs that the specific immune response was even increased by the chemotherapy.

The vaccine administration regimen according to the invention contains preferably not only the initial vaccination within the scope of the chemotherapy, but also several booster vaccinations at certain time intervals which for practical reasons possibly can be equal to the intervals of the chemotherapy. Also following the chemotherapy, the long-term immunotherapy over months and years very much is a suitable regimen. Both, the initial vaccination and also later booster vaccinations preferably are effected with the same vaccine.

The combination with the adjuvant or palliative chemotherapy is preferred. The combination with a monotherapy or polytherapy is possible. For reasons of the different mechanisms of action, the vaccine preferably is combined with the polychemotherapy.

Preferable agents used for the chemotherapy are alkylating pharmaceutical preparations. Thus, e.g., agents containing taxane, anthracyclins or platinum are preferred. All the conventional preparations which are used for various cancer treatments can be combined according to the invention. The chemotherapeutic agents commonly are administered intravenously or perorally. Peroral forms of administration of the chemotherapeutic agents may possibly also be administered with the peroral form of the vaccine according to the invention as a combination preparation.

By the vaccine employed according to the invention, a functional immune response is induced, which is mainly carried by the humoral immune system. As immediate reaction, e.g., an increased immunoglobulin titer is found in the patient's serum. Mainly those antibodies occur in the serum which are specific of the vaccination antigen. A cellular immune response can be detected with the common test systems. In any case, it can be proven that tumor cells can be specifically bound. The number of tumor cells found in blood or in the bone marrow shall even be reducible by the specific immune response.

A vaccination as defined by the present invention may basically be carried out both for a therapeutic and also for a prophylactic treatment (as with all anti-microbial vaccines). This means that the inventive vaccination against cancer may be understood both as a therapeutic and also as a prophylactic application. Thus, by vaccinating persons who do not suffer from cancer with suitable antibodies, possibly a prophylactic protection against the emergence of cancer-associated conditions, in particular the formation of metastases, can be achieved. Such a prophylactic vaccination particularly—though not exclusively—aims at persons who run an increased, possibly genetically caused, risk of developing a cancer disease.

In a preferred embodiment, the vaccine used according to the invention comprises a human cellular membrane antigen or an antibody against this membrane protein, or a corresponding anti-idiotypic antibody, respectively. Such a membrane protein plays a role in adhesion processes. Adhesion processes preferably are cell-cell interactions, in which ligands, or receptors, respectively, on the cell surface are involved. Accordingly, adhesion molecules are ligands, or receptors, respectively, at the cell surface which serve the cell-cell interaction. A subgroup of such adhesion molecules are the self-adhesion molecules. They have the property of being able to bind to themselves.

The physiologic effect of an immune response induced by a vaccination with an antibody against a TAA naturally will depend on the function of the respective TAA. If the TAA has, e.g., a function as a receptor for the adhesion of tumor cells, in particular to a ligand on endothelium cells of the vessel system (such a property is important for the ability of disseminated cancer cells of leaving the vessel system and establishing themselves in tissue so as to form a metastasis there), then this adhesion ability will be lowered by vaccination with a suitable antibody against this TAA, because in circulation and in the tissue permanently induced antibodies are present which compete the interaction of the TAA with its ligand, since they mimic the TAA in soluble form.

Thus, according to the above discussion, by vaccinating with suitable TAA or with corresponding antibodies which have a function for the malignancy of tumor cells it can be achieved that the induced immune response interferes with the function of the TAA in its interaction with its ligand, making the latter more difficult or preventing the latter. This means that cancer cells do not or cannot sufficiently exhibit properties which are important for the malignant phenotype, whereby the progress of the disease can be slowed or stopped, and particularly in the early stages, the formation of metastases can be suppressed and in the late stage of metastasizing cancer, the metastases can be reduced.

In a further preferred embodiment, the cellular membrane antigen is capable of self-adhesion, i.e. certain epitopes of the antigen are responsible for the homophilic binding with an equal antigen on another cell. Examples of such antigens are, i.a., N-CAM (neuronal cellular adhesion molecule), CEA (carcino embryonic antigen) and Ep-CAM (epithelial cell adhesion molecule). Antibodies directed against epitopes of self-adhesion antigens which are involved in this function, according to the above discussion can carry a complementary structure information of such an epitope. Therefore, according to the above discussion, by vaccination with such vaccinating antigens, the formation of antibodies can be induced which carry the properties of this self-adhesion in the binding reaction. This means that such induced antibodies in turn can bind to the self-adhesion antigen, since in such an instance, receptor and ligand are identical. Thus, by vaccinating cancer patients, possibly with suitable antibodies against self-adhesion antigens, an immune response can be induced which in turn binds directly to tumor cells and thereby triggers manifold therapeutic effects. On the one hand, the ability of self-adhesion, which is important for malignant cells, is blocked, and, on the other hand, by the induced antibodies' binding to the cancer cells, human effector functions, such as complement-dependent lysis and/or lysis by activation of cytotoxic effector cells, can be triggered which will lead to the destruction of the cancer cells.

By all the above-mentioned mechanisms and effects, the vaccination of cancer patients with suitable TAAs or corresponding antibodies against TAAs or anti-idiotypic antibodies, can suppress the formation of new metastasis and at least slow down the dissemination of the disease. In the early stages of the disease, e.g., shortly before or after successful surgery of a primary tumor (adjuvant stage), by such vaccinations residual, disseminated tumor cells will be prevented from establishing themselves as new metastases. By the inventive combination with chemotherapy, the dividing active tumor cells are killed, on the one hand; on the other hand, the relapse-free life span and, thus, also the total survival time of such patients can be extended by the targeted immunotherapy. By such vaccinations and booster vaccinations carried out at suitable intervals, possibly a life-long protection against the formation of metastases can be obtained. What is of particular interest are vaccinations of cancer patients with suitable TAAs or corresponding antibodies against a self-adhesion TAA, since in these instances, as described above, an increased therapeutic effect is possible by an additional direct attack of the induced immune response on tumor cells.

Methods of finding suitable antigenic structures, modelling and preparing TAA-derived peptides, polypeptides or proteins, or nucleic acids coding therefor, furthermore, lipoproteins, glycolipids, carbohydrates or lipids, are known to the person skilled in the art and can be provided without any undue experimental expenditures for the respective tumor-specific structure. Furthermore, the methods of formulating a vaccine suitable for the inventive method are known.

The TAAs, their derivatives, epitopes and mimics can be obtained from natural or synthetic sources. Also the antibody components can be chemically synthesized and subsequently connected with epitope structures, or synthesized in common. In a chemical synthesis of antibody carrier molecules it is possible to introduce reactive groups at special sites so as to be able both to control the extent of coupling with an epitope as well as the type and location of the binding.

Immunogenic TAAs, their epitopes, mimics or antibodies, may also be prepared as recombinant molecules by genetic engineering methods. By changing by genetical engineering nucleic acids which encode native molecules, e.g. suitable derivatives can be produced. A glycosylation of a recombinant gene product with corresponding tumor-associated glycan structures can also be effected by a production in cells which have been genetically modified such that they will glycosylate proteins accordingly. Such cells may be natural isolates (cell clones), they can be found by an appropriate screening for the desired glycosylation. Yet, also cells may be modified such that they will express the corresponding enzymes required for the desired glycosylation such that particularly the desired glycosylation will be found on the recombinant polypeptide or protein (Glycoconj. J. (1999), 16: 81). However, it is also possible to enzymatically produce, or change, respectively, the glycosylation pattern of proteins (Clin. Chem. Lab. Med. (1998), 36: 373).

According to a particular embodiment of the present invention, the vaccine used according to the invention comprises a nucleic acid molecule as a mimic for a TAA, wherein the nucleic acid molecule encodes a proteinaceous TAA as defined by the present invention. The DNA vaccine obtained is administered just like tumor vaccines on protein base.

The present invention also relates to a kit which is suitable for the prophylactic and/or therapeutic treatment of cancer-associated conditions. This kit comprises
a) a vaccine based on a tumor-associated antigen, its epitope, mimotope, specific or anti-idiotypic antibody, and
b) an agent for chemotherapy.

The selection of the components of the kit according to the invention as well as their combination is carried out as described before.

Preferably, the kit further comprises suitable application devices, such as, e.g., syringes, infusion devices, etc. If the vaccine is provided in lyophilized form, the kit will further contain a suitable reconstituting solution which optionally comprises special stabilizers or reconstitution accelerators. The further preferred kit comprises several units of the vaccine used according to the invention, which will be employed for the initial vaccination as well as for one or more, preferably up to three, booster vaccinations.

The number of booster vaccinations may, however, also be higher, and for this, kits containing several vaccination units alone, without a combination with a chemotherapeutic agent, are offered. The frequency optionally will range from 1 to 12 per year, particularly preferred it will range from 4 to 8 per year. The dosage may remain equal or may decrease.

Booster vaccinations can be carried out at regular intervals, in principle life-long. Suitable intervals range from about 6 to 24 months and can be determined by checking the titer of the induced antibodies (a booster should be made as soon as the titers of the induced antibodies have markedly dropped).

In the following, clinical trials are described, demonstrating that the vaccination of patients immediately before chemotherapy with a certain murine MAB (HE2) which is directed against the self-adhesion-TAA Ep-CAM, directly leads to the induction of antibodies which selectively bind to the Ep-CAM molecule. This immune response is at least as good in combination with chemotherapy as it is without chemotherapy. There is even a tendency towards an improvement by the simultaneous chemotherapy. This shows by way of example, yet without any restriction, that by vaccinating with suitable TAA, or with antibodies against a TAA, or their derivatives which contain at least the idiotype of the starting antibody, an immune response is induced which can be combined with the chemotherapy.

The murine monoclonal antibody HE2 was generated therefor, according to standard methods of hybridoma technology described per se (cf. WO 00/41722). Balb/c mice were immunized with human colorectum cancer cells according to standard protocols. The spleen cells were fused with the mouse myeloma line P3X63Ag8, and hybridomas were selected which produce antibodies that bind selectively to human colorectal cancer cells, yet not to melanoma cells. Finally, a hybridoma was chosen which secretes an IgG2a/kappa antibody. This antibody (HE2) binds to Ep-CAM, as can be shown e.g. by Western blot analysis with membrane preparations of KTO III stomach cancer cells, in comparison to a known anti-Ep-CAM antibody (KS1-4).

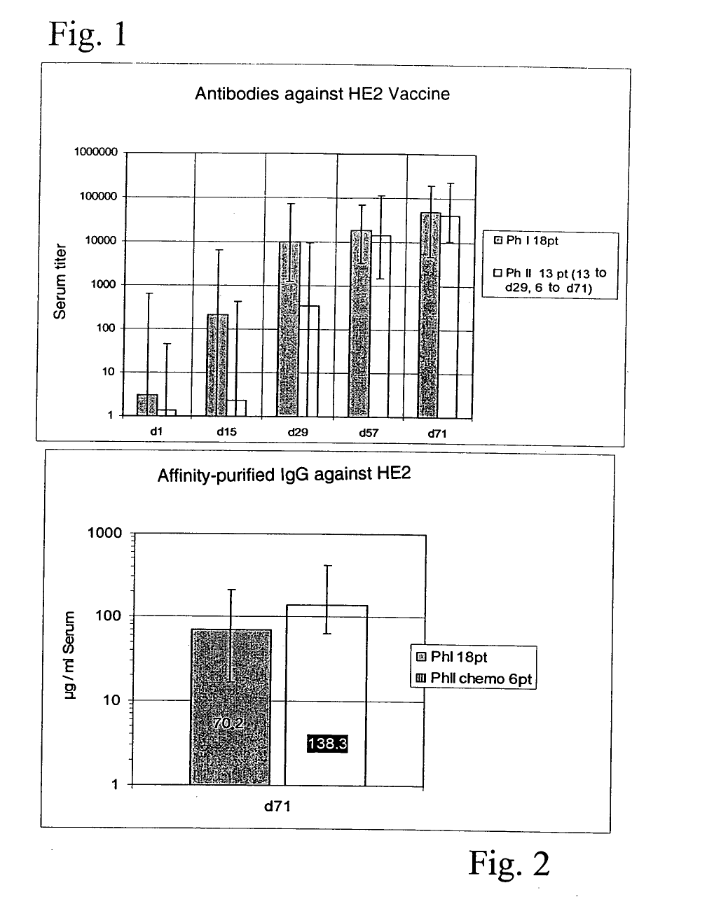

FIG. 1 shows the immune serum globulin content from patients who had been vaccinated with HE2 with and without simultaneous chemotherapy.

FIG. 2 shows the content of IgG, directed against HE2 in serum.

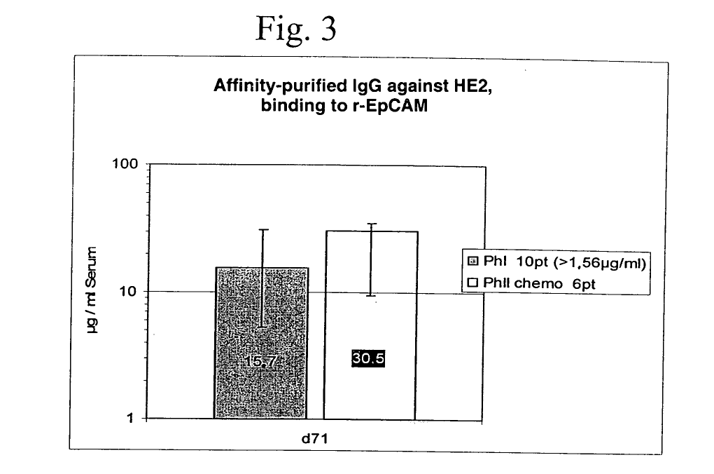

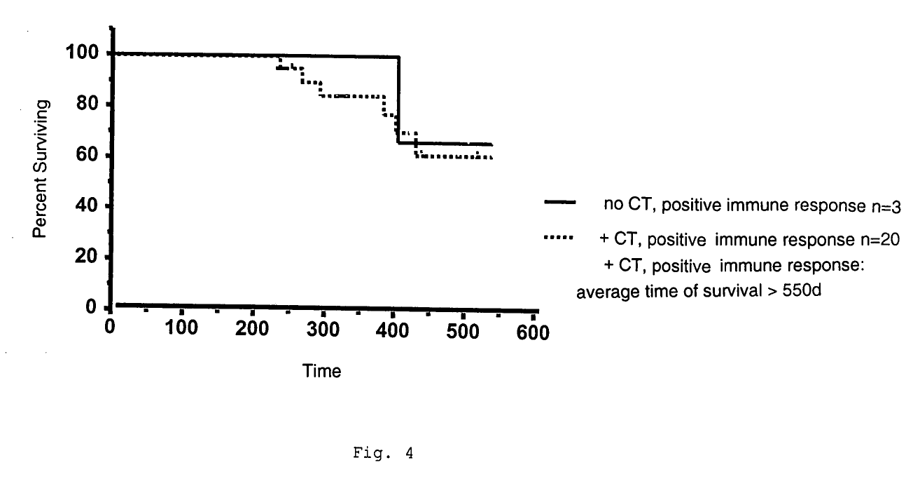

FIG. 3 shows the content of IgG, directed against HE2 in serum specifically binding to recombinant Ep-CAM.

FIG. 4 shows the influence of chemotherapy and vaccination on the survival rate in patients suffering from colorectal cancer, stage 4.

The following examples shall further illustrate the present invention, without, however, restricting it.

### EXAMPLES

### Example 1

### Treatment of Cancer Patients with HE2 in Combination with Chemotherapy

Patients afflicted with a metastasizing, epithelial cancer were subcutaneously treated both by chemotherapy and also by immunotherapy, by an active immunization with a vaccine containing HE2. The patients received an initial vaccination on day 1 of the chemotherapy, at least 1 hour prior to the treatment with the chemotherapeutic agent. The chemotherapy was an adjuvant or palliative one.

On days 1, 15, 29, 57 and 71, serum was taken from the patients so as to determine the immune response. The results of the determinations are illustrated in FIGS. 1 to 3.

By the simultaneous chemotherapy, at least no deterioration of the immune response was found. A tendency for an even somewhat increased specific immune response could be observed.

### Example 2

### Treatment of Cancer Patients with HE2 in Combination with Chemotherapy

Patients afflicted with metastasizing, epithelial cancer (colorectal cancer, stage 4) were subcutaneously treated both by chemotherapy and also by immunotherapy, by active immunization with a vaccine containing HE2; the immunization was effected on days 1, 15, 29 and 57; subsequently, the vaccinations were repeated every 4 months. The chemotherapy was an adjuvant or a palliative one.

As the primary end point of the study, the survival rate was determined; the results are illustrated in FIG. 4 (n is the number of patients, CT is chemotherapy). It has been shown that both with and also without chemotherapy, the survival time was markedly increased by the treatment with the vaccine. While in patients who received chemotherapy only, the survival rate after one year was at approximately 44%, in patients with chemotherapy and an immune response to the administration of the vaccine, a survival rate of more than 80% was found after one year.

1. The use of a vaccine based on a tumor-associated antigen, its epitope, mimotope, specific or anti-idiotypic antibody, for the preparation of a medicament for the prophylactic and/or therapeutic active immunization against cancer in combination with chemotherapy.

2. The use according to claim 1, characterized in that the tumor-associated antigen is selected from the group of peptides or proteins, T-cell peptides, carbohydrates, glycolipids and epitopes or mimotopes of these antigens.

3. The use according to claim 1 or 2, characterized in that the vaccine comprises an antibody or an antibody derivative directed against a tumor-associated antigen.

4. The use according to claim 1 or 2, characterized in that the vaccine comprises an anti-idiotypic antibody or its derivative, directed against antibodies, specifically for a tumor-associated antigen.

5. The use according to any one of claims 1 to 4, characterized in that the vaccine is administered before and/or during chemotherapy.

6. The use according to claim 5, characterized in that the vaccine is even further administered after the chemotherapy.

7. The use according to any one of claims 1 to 6, characterized in that an adjuvant or palliative chemotherapy is carried out.

8. The use according to any one of claims 1 to 7, characterized in that a polychemotherapy is carried out.

9. The use according to any one of claims 1 to 8, characterized in that the chemotherapy is carried out with an agent comprising taxane, anthracyclin or platinum.

10. The use according to any one of claims 1 to 9, characterized in that a functional immune response against tumor cells is induced.

11. The use according to any one of claims 1 to 10, characterized in that disseminated tumor cells are reduced.

12. The use according to any one of claims 1 to 11, for the prophylactic and/or therapeutic treatment of cancer-associated conditions, in particular the formation of metastases.

13. The use according to any one of claims 1 to 12, for the prophylactic and/or therapeutic treatment of epithelial cancer.

14. The use according to any one of claims 1 to 13, characterized in that the medicament can be administered with an adjuvant without particular side effects.

15. A kit for the prophylactic and/or therapeutic treatment of cancer-associated conditions, containing
a) a vaccine based on a tumor-associated antigen, its epitope, mimotope, specific or anti-idiotypic antibody, and
b) an agent for chemotherapy.

16. A kit according to claim 15, characterized in that the tumor-associated antigen is selected from the group of peptides or proteins, T-cell peptides, carbohydrates, glycolipids and epitopes or mimotopes of these antigens.

17. A kit according to claim 15, characterized in that the vaccine comprises an antibody directed against a tumor-associated antigen.

18. A kit according to claim 15, characterized in that the vaccine comprises an anti-idiotypic antibody directed against an antibody specific for a tumor-associated antigen.

19. A kit according to any one of claims 15 to 18, characterized in that the vaccine contains the tumor-associated antigen, its epitope, mimotope, specific or anti-idiotypic antibody in an immunogenic amount of from 0.01 μg to 10 mg.

20. A kit according to any one of claims 15 to 19, characterized in that the vaccine comprises at least one vaccine adjuvant.

21. A kit according to any one of claims 15 to 20, characterized in that the vaccine is provided in a formulation suitable for subcutaneous, intramuscular, intradermal, transdermal or mucosal, such as nasal or peroral, administration.

22. A kit according to any one of claims 15 to 21, characterized in that the agent for chemotherapy comprises taxane, anthracyclin or platinum.

23. A kit according to any one of claims 15 to 22, characterized in that the agent for chemotherapy is provided in a formulation suitable for intravenous or peroral administration.

24. A kit according to any one of claims 15 to 23, characterized in that several units of the vaccine are contained therein.
